(19) United States
(12) Patent Application Publication
(10) Pub. No.: US 2014/0335031 A1
(43) Pub. Date: Nov. 13, 2014

(54) LIQUID SOLUTION COMPRISING PEBA

(71) Applicant: ARKEMA FRANCE, Colombes Cedex (FR)

(72) Inventors: Frédéric Malet, Rouen (FR); Marie-Ange Michel, Granchain (FR)

(21) Appl. No.: 14/359,923

(22) PCT Filed: Jan. 4, 2013
(86) PCT No.: PCT/FR2013/050019

§ 371 Date: May 22, 2014

(30) Foreign Application Priority Data
Jan. 5, 2012 (FR) 1250119

(51) Int. Cl. A61K 8/88; A61Q 17/04; A61Q 1/02; A61Q 13/00; C08K 5/134; A61K 8/04; C09D 177/00; C08K 5/05; C08K 5/20; C08K 5/315; A61K 47/34; A61K 9/08

(52) U.S. Cl. CPC A61K 8/88; A61K 47/34; A61Q 17/04; A61Q 1/02; A61Q 13/00; A61K 9/08; A61K 8/04; C09D 177/00; C08K 5/05; C08K 5/20; C08K 5/315; C08K 5/1345; A61K 2800/262; A61K 2800/10; A61K 2800/40
USPC 424/59; 514/772.3; 424/63; 512/2; 524/391; 523/105; 524/219; 427/427.4; 427/430.1; 427/429

(57) ABSTRACT

A liquid composition including between 1 and 15 parts by weight of PEBA to 100 parts by weight of solvent, wherein: said solvent includes between 1 and 49% by volume of alcohol low in C₁ to C₄ and between 51 and 99% by volume of a cosolvent miscible with alcohol, in the total volume of solvent. Further, use of such a composition for producing water-resistant objects, such as coatings, films, varnishes, paints, in particular anti-graffiti coatings, seals, in particular sealing joints, personal protective equipment, gloves, flexible masks, condoms, and protective equipment for electronic or computer hardware.

### TECHNICAL FIELD

An embodiment of the disclosure is a composition comprising a thermoplastic elastomer polymer, in particular a copolymer comprising polyether blocks and polyamide blocks (hereinafter PEBA).

An embodiment of the disclosure relates in particular to a liquid solution, preferably a clear liquid solution, comprising at least one PEBA and to the use of this solution in particular in the manufacture of water-resistant coatings.

### SUMMARY

Thermoplastic polymers are generally provided in the form of granules or powders. Consequently, the formulation with these powders generally requires intermediate stages of milling, indeed even of sieving, in the form of a fine powder with a D50 generally of less than 10 or 20 μm, and of prior dispersion of this powder in the liquid in order for the texture of the final product incorporating the powder to exhibit perfect homogeneity and a uniform appearance. Furthermore, the high volatility of fine powders means that their use in formulations requires many precautions. It is in particular difficult for formulators to precisely and reproducibly quantify the powder content of the formulations.

Among thermoplastic elastomer polymers, copolymers comprising polyether blocks and polyamide blocks (hereinafter PEBAs) are known for their great flexibility (a very broad range), their elasticity, chemical resistance and temperature resistance, their dynamic properties, their stable properties at temperatures from −40° C. to 80° C., their waterproof/breathable properties, their resistance to UV radiation, and the like.

These properties are already being made use of in applications as varied as sports equipment, spectacles, textiles, the motor vehicle industry or the coating of materials by fine PEBA powders. On the other hand, these properties of PEBAs have never been made use of in the field of liquid formulations, in particular those used in coatings or the manufacture of films, paints, varnishes, gloves or cosmetics.

An aim of embodiments of the disclosure is thus to provide ready-for-use compositions comprising PEBA which facilitate the processing of the PEBA by the formulators by being usable directly (by simple incorporation) in paint, coating or cosmetic formulations, for example in order to confer on them properties of persistence or of water resistance. Thus, the formulator no longer needs to adjust beforehand the form of the PEBAs, sold commercially in the powder or granule form. Furthermore, the PEBA contents in the formulations can be easily quantified and reproduced.

It is an aim in particular of embodiments of the disclosure to provide such PEBA compositions in the ready-for-use liquid form.

The applicant company has shown that it is possible to manufacture, starting from PEBA, a liquid solution which remains stable, indeed even clear, at ambient temperature, that is to say at a temperature of 15 to 25° C.

### DETAILED DESCRIPTION

An embodiment of the disclosure is thus a composition comprising: from 1 to 15 parts by weight of PEBA per 100 parts by weight of solvent, characterized in that said solvent comprises from 1% to 49%, preferably from 10% to 45%, preferably from 15% to 40%, preferably from 20% to 40%, by volume of light C₁ to C₆ alcohol and from 51% to 99%, preferably from 55% to 90%, preferably from 60% to 85%, preferably from 60% to 80%, by volume of a cosolvent which is miscible with the alcohol, with respect to the total volume of solvent.

The term “miscible” is understood to mean the ability of liquids to mix. If the mixture obtained is homogeneous, the liquids are described as miscible. Conversely, the liquids are said to be immiscible if they cannot mix and they form a heterogeneous mixture: several phases are then observed.

The cosolvent is advantageously chosen from fatty acids, fatty esters, fatty alcohols, fatty ethers and their mixtures.

Advantageously, the composition according to embodiments of the disclosure is provided in the form of a solution which is clear at ambient temperature, i.e. a temperature of 15 to 25° C. The notion of clearness contrasts with the notion of opaqueness. It is thus possible to refer to % of transmission of light at a visible wavelength, typically 560 nm. The term “clear solution” is understood to mean, within the meaning of the disclosure, a solution exhibiting, at 560 nm, a transmission of greater than 50%, preferably greater than 70%, and the term “opaque solution” is understood to mean a solution exhibiting a transmission of less than or equal to 50%, preferably of less than or equal to 70%. Preferably, within the meaning of the disclosure, the mixture of two miscible liquids forms a clear homogeneous solution.

Preferably, the composition comprises from 1 to 10 parts, preferably from 2 to 8 parts, preferably from 4 to 6 parts, of PEBA, per 100 parts of solvent.

The “copolymers comprising polyether blocks and polyamide blocks”, abbreviated to “PEBAs”, result from the polycondensation of polyamide blocks comprising reactive ends with polyether blocks comprising reactive ends, such as, inter alia:

1) polyamide blocks comprising diamine chain ends with polyoxyalkylene blocks comprising dicarboxyl chain ends;

2) polyamide blocks comprising dicarboxyl chain ends with polyoxyalkylene blocks comprising diamine chain ends, which are obtained by cyanoethylation and hydrogenation of aliphatic α,ω-dihydroxylated polyoxyalkylene blocks, known as polyetherdiols;

3) polyamide blocks comprising dicarboxyl chain ends with polyetherdiols, the products obtained being, in this specific case, polyetheresteramides.

The polyamide blocks comprising dicarboxyl chain ends originate, for example, from the condensation of precursors of polyamides in the presence of a chain-limiting dicarboxylic acid. The polyamide blocks comprising diamine chain ends originate, for example, from the condensation of precursors of polyamides in the presence of a chain-limiting diamine.

The number-average molar mass Mn of the polyamide blocks is between 400 and 20 000 g/mol, preferably between 500 and 10 000 g/mol.

The polymers comprising polyamide blocks and polyether blocks can also comprise randomly distributed units.

Use may be advantageously made of three types of polyamide blocks.

According to a first type, the polyamide blocks originate from the condensation of a dicarboxylic acid, in particular those having from 4 to 20 carbon atoms, preferably those having from 6 to 18 carbon atoms, and of an aliphatic or aromatic diamine, in particular those having from 2 to 20 carbon atoms, preferably those having from 6 to 14 carbon atoms.

Mention may be made, as examples of dicarboxylic acids, of 1,4-cyclohexanedicarboxylic acid, butanedioic, adipic, azelaic, suberic, sebacic, dodecanedicarboxylic and octadecanedicarboxylic acids and terephthalic and isophthalic acids, but also dimerized fatty acids.

Mention may be made, as examples of diamines, of tetramethylenediamine, hexamethylenediamine, 1,10-decamethylenediamine, dodecamethylenediamine, trimethylhexamethylenediamine, the isomers of bis(4-aminocyclohexyl)methane (BACM), bis(3-methyl-4-aminocyclohexyl)methane (BMACM), and 2,2-bis(3-methyl-4-aminocyclohexyl)propane (BMACP), and di(para-aminocyclohexyl)methane (PACM), and isophoronediamine (IPDA), 2,6-bis(aminomethyl)norbornane (BAMN) and piperazine (Pip).

The following blocks advantageously exist: PA4.12, PA4.14, PA4.18, PA6.10, PA6.12, PA6.14, PA6.18, PA9.12, PA10.10, PA10.12, PA10.14 and PA10.18.

According to a second type, the polyamide blocks result from the condensation of one or more α,ω-aminocarboxylic acids and/or of one or more lactams having from 6 to 12 carbon atoms in the presence of a dicarboxylic acid having from 4 to 12 carbon atoms or of a diamine. Mention may be made, as examples of lactams, of caprolactam, enantholactam and lauryllactam. Mention may be made, as examples of α,ω-aminocarboxylic acid, of aminocaproic, 7-aminoheptanoic, 11-aminoundecanoic and 12-aminododecanoic acids.

Advantageously, the polyamide blocks of the second type are of polyamide 11, of polyamide 12 or of polyamide 6.

According to a third type, the polyamide blocks result from the condensation of at least one α,ω-aminocarboxylic acid (or one lactam), at least one diamine and at least one dicarboxylic acid.

In this case, the polyamide PA blocks are prepared by polycondensation:
of the linear aliphatic or aromatic diamine or diamines having X carbon atoms;
of the dicarboxylic acid or acids having Y carbon atoms; and
of the comonomer or comonomers {Z} chosen from the lactams and the α,ω-aminocarboxylic acids having Z carbon atoms and the equimolar mixtures of at least one diamine having X1 carbon atoms and of at least one dicarboxylic acid having Y1 carbon atoms, (X1, Y1) being different than (X, Y),
said comonomer or comonomers {Z} being introduced in a proportion by weight ranging up to 50%, preferably up to 20% and more advantageously still up to 10%, with respect to the combined polyamide precursor monomers;
in the presence of a chain-limiting agent chosen from dicarboxylic acids.

Use is advantageously made, as chain-limiting agent, of the dicarboxylic acid having Y carbon atoms, which is introduced in excess with respect to the stoichiometry of the diamine or diamines.

According to an alternative form of this third type, the polyamide blocks result from the condensation of at least two α,ω-aminocarboxylic acids or of at least two lactams having from 6 to 12 carbon atoms or of a lactam and of an aminocarboxylic acid not having the same number of carbon atoms, in the optional presence of a chain-limiting agent. Mention may be made, as examples of aliphatic α,ω-aminocarboxylic acid, of aminocaproic, 7-aminoheptanoic, 11-aminoundecanoic and 12-aminododecanoic acids. Mention may be made, as examples of a lactam, of caprolactam, enantholactam and lauryllactam. Mention may be made, as examples of aliphatic diamines, of hexamethylenediamine, dodecamethylenediamine and trimethylhexamethylenediamine. Mention may be made, as example of cycloaliphatic diacids, of 1,4-cyclohexanedicarboxylic acid. Mention may be made, as examples of aliphatic diacids, of butanedioic, adipic, azelaic, suberic, sebacic and dodecanedicarboxylic acids, dimerized fatty acids (these dimerized fatty acids preferably have a dimer content of at least 98%; preferably, they are hydrogenated; they are sold under the PRIOPOL® trade name by Uniqema or under the EMPOL® trade name by Henkel) and polyoxyalkylene-α,ω-diacids. Mention may be made, as examples of aromatic diacids, of terephthalic (T) and isophthalic (I) acids. Mention may be made, as examples of cycloaliphatic diamines, of the isomers of bis(4-aminocyclohexyl)methane (BACM), bis(3-methyl-4-aminocyclohexyl)methane (BMACM), and 2,2-bis(3-methyl-4-aminocyclohexyl)propane (BMACP), and di(para-aminocyclohexyl)methane (PACM). The other diamines commonly used can be isophoronediamine (IPDA), 2,6-bis(aminomethyl)norbornane (BAMN) and piperazine.

Mention may be made, as examples of polyamide blocks of the third type, of the following:
6.6/6 in which 6.6 denotes hexamethylenediamine units condensed with adipic acid and 6 denotes units resulting from the condensation of caprolactam.
6.6/6.10/11/12 in which 6.6 denotes hexamethylenediamine condensed with adipic acid, 6.10 denotes hexamethylenediamine condensed with sebacic acid, 11 denotes units resulting from the condensation of aminoundecanoic acid and 12 denotes units resulting from the condensation of lauryllactam.

The polyether blocks can represent from 5% to 85% by weight of the copolymer comprising polyamide and polyether blocks. The mass Mn of the polyether blocks is between 100 and 6000 g/mol and preferably between 200 and 3000 g/mol.

The polyether blocks consist of alkylene oxide units. These units can, for example, be ethylene oxide units, propylene oxide units or tetrahydrofuran units (which results in the polytetramethylene glycol sequences). Use is thus made of PEG (polyethylene glycol) blocks, that is to say those consisting of ethylene oxide units, PPG (polypropylene glycol) blocks, that is to say those consisting of propylene oxide units, PO3G (polytrimethylene glycol) blocks, that is to say those consisting of polytrimethylene ether glycol units (such copolymers with polytrimethylene ether blocks are described in the document U.S. Pat. No. 6,590,065), and PTMG blocks, that is to say those consisting of tetramethylene glycol units, also known as polytetrahydrofuran blocks. The PEBA copolymers can comprise several types of polyethers in their chain, it being possible for the copolyethers to be block or random copolyethers. Preferably, the composition according to embodiments of the disclosure comprises a PEBA, the polyether blocks of which are composed predominantly based on PTMG, that is to say at more than 50% by weight with respect to the total weight of polyether blocks. This is because it has been found, surprisingly, that these PTMG-based PEBAs dissolve very easily in the solvent according to embodiments of the disclosure. Preferably, the PEBA used in the composition of embodiments of the disclosure comprises more than 50% by weight, preferably more than 70% by weight, indeed even more than 75% by weight, of polyether blocks with respect to the total weight of PEBA. This is because it has been found that these PEBAs having a high percentage by weight of polyether blocks are easier to dissolve than the PEBAs having a high percentage by weight of polyamide blocks.

Use may also be made of blocks obtained by oxyethylation of bisphenols, such as, for example, bisphenol A. The latter products are described in the patent EP 613 919.

The polyether blocks can also consist of ethoxylated primary amines. Mention may be made, as examples of ethoxylated primary amines, of the products of formula:

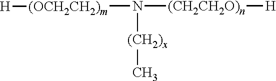

in which m and n are between 1 and 20 and x is between 8 and 18. These products are commercially available under the NORAMAX® trade name from CECA and under the GENAMIN® trade name from Clariant.

The soft polyether blocks can comprise polyoxyalkylene blocks comprising NH₂ chain ends, it being possible for such blocks to be obtained by cyanoacetylation of aliphatic α,ω-dihydroxylated polyoxyalkylene blocks, known as polyetherdiols. More particularly, use may be made of Jeffamines (for example, JEFFAMINE® D400, D2000, ED 2003 or XTJ 542, commercial products from Huntsman, also described in the documents of patents JP 2004346274, JP 2004352794 and EP 1 482 011).

The polyetherdiol blocks are either used as is and copolycondensed with polyamide blocks comprising carboxyl ends or they are aminated in order to be converted into polyetherdiamines and condensed with polyamide blocks comprising carboxyl ends. The general method for the two-stage preparation of PEBA copolymers having ester bonds between the PA blocks and the PE blocks is known and is described, for example, in the French patent FR 2 846 332. The general method for the preparation of the PEBA copolymers of embodiments of the disclosure having amide bonds between the PA blocks and the PE blocks is known and described, for example, in the European patent EP 1 482 011. Polyether blocks may also be mixed with polyamide precursors and a chain-limiting diacid in order to prepare polymers comprising polyamide blocks and polyether blocks having randomly distributed units (one-stage process).

Of course, the designation PEBA in the disclosure relates equally well to the PEBAX® products sold by Arkema, to the VESTAMID® products sold by EVONIK®, to the GRILAMID® products sold by EMS, to the KELLAFLEX® products sold by DSM or to any other PEBA from other suppliers.

Advantageously, the PEBA copolymers have PA blocks of PA6, of PA11, of PA12, of PA6.12, of PA6.6/6, of PA10.10 and/or of PA6.14, preferably PA11 and/or PA12 blocks; and PE blocks of PTMG, of PPG and/or of PO3G. The PEBAs based on PE blocks consisting predominantly of PEG are to be ranked in the range of the hydrophilic PEBAs. The PEBAs based on PE blocks consisting predominantly of PTMG are to be ranked in the range of the hydrophobic PEBAs.

Advantageously, said PEBA used in the composition according to embodiments of the disclosure is obtained, at least partially, from bioresourced starting materials.

Starting materials of renewable origin or bioresourced starting materials is understood to mean substances which comprise bioresourced carbon or carbon of renewable origin. Specifically, unlike the substances resulting from fossil materials, the substances composed of renewable starting materials comprise ¹⁴C. The “content of carbon of renewable origin” or “content of bioresourced carbon” is determined by application of the standards ASTM D 6866 (ASTM D 6866-06) and ASTM D 7026 (ASTM D 7026-04). By way of example, the PEBAs based on polyamide 11 originate at least in part from bioresourced starting materials and exhibit a content of bioresourced carbon of at least 1%, which corresponds to a ¹²C/¹⁴C isotopic ratio of at least 1.2×10⁻¹⁴. Preferably, the PEBAs according to embodiments of the disclosure comprise at least 50% by weight of bioresourced carbon with respect to the total weight of carbon, which corresponds to a ¹²C/¹⁴C isotopic ratio of at least 0.6×10⁻¹². This content is advantageously higher, in particular up to 100%, which corresponds to a ¹²C/¹⁴C isotopic ratio of 1.2×10⁻¹², in the case of PEBAs comprising PA11 blocks and PE blocks comprising PO3G, PTMG and/or PPG resulting from starting materials of renewable origin.

The term “C₁ to C₆ alcohol” is understood to mean the alcohols for which the number of carbons of the carbon-based chain does not exceed 6 and which are water-soluble, such as ethanol or isopropanol. Alcoholic solutions obtained by simple mixing of these alcohols of water can also be used in the composition; as well as glycols, such as ethylene glycol or propylene glycol; or polyols, such as glycerol or glycerine, sorbitol or sorbitol syrup. Mention may also be made of butanediols, for example 1,3- and 1,4-butanediols. In addition, aromatic alcohols, such as meta-cresol or benzyl alcohol, can be used but are less preferred for HSE reasons.

The fatty alcohols within the meaning of embodiments of the disclosure are alcohols for which the carbon-based chain comprises at least 7 carbons, preferably from 7 to 10 carbons, so that they are liquid at ambient temperature. The presence of the hydroxyl confers on them an immiscibility with the C₁ to C₆ alcohol. Mention may in particular be made of benzyl alcohol, which acts as solvent and preservative.

The fatty acids within the meaning of embodiments of the disclosure are organic acids which occur in lipids. Their carbon-based chain is more or less lengthy (from C₄ to C₃₀) and they can be saturated or unsaturated. The saturated fatty acids are solid at ambient temperature (25° C.), except for the C₄ and C₆ acids. The unsaturated fatty acids are liquid. Mention may be made, by way of example, of lauric acid, stearic acid or oleic acid.

The fatty esters result either from the combination of a fatty acid with a short-chain alcohol (for example, isopropyl palmitate, isopropyl myristate or diisopropyl sebacate, which form liquid fatty esters), or from the combination of a fatty acid with a fatty alcohol (for example, isostearyl isostearate), or from the combination of a short-chain acid with a fatty alcohol comprising a more or less lengthy chain (for example, benzoic acid with a C₁₂-C₁₅ fatty alcohol, forming benzoates of fatty alcohols, C₁₂-C₁₅ alkyl benzoate). Mention may also be made, as examples of fatty esters which can be used in the compositions according to embodiments of the disclosure, of: esters of benzoic acid, such as the 2-phenylethyl ester of benzoic acid, esters of salicylic acid, such as ethylhexyl salicylate, fatty esters of acrylic acid, such as 2-ethylhexyl 2-cyano-3,3-diphenylacrylate; or also isopropyl lauroyl sarcosinate, isononyl isononanoate, dicaprylyl carbonate, and the like.

Advantageously, the composition according to embodiments of the disclosure additionally comprises from 1 to 10 parts of additive chosen from: UV screening agents, antioxidants, pigments, fillers, such as talc, nylon or silica, or cosmetic or pharmaceutical active agents, and any other agent which can also participate in the composition of a coating, of a varnish, of a transparent flexible film or of a cosmetic or pharmaceutical product for improving the texture, the spreading, the feel, the appearance or the stability of said composition.

The liquid composition according to embodiments of the disclosure is in particular easily manufactured by simple mixing, such as mixing at reflux, of the PEBA in ethanol and the co-solvent, preferably by heating until all the components have completely dissolved.

Another embodiment of the disclosure is the use of the composition according to embodiments of the disclosure in the manufacture of a product having at least one of the following forms: dispersion, solution, emulsion, microemulsion, nanoemulsion, dry emulsion, suspension, aerosol, gel, in particular compact gel, gum, plastic gum, paste, foam, cream, powder, such as loose powder, compact powder or expanded powder, butter, film, elastic film and their mixtures or combinations.

The composition according to embodiments of the disclosure can advantageously be used in the manufacture of water-resistant objects, such as coatings, films, varnishes, paints, in particular anti-graffiti coatings, seals, in particular leak-tight seals, individual protective equipment, gloves, flexible masks, condoms, protective accessories for electronic or computing equipment, and any other application requiring a flexible watertight film.

Advantageously, said objects are manufactured by at least one of the following methods: spreading, spraying, over a mold or any support, in particular made of wood, plastic or metal, the skin, the nails, and the like, by dipping, evaporation of the solvent from the composition.

The composition according to embodiments of the disclosure can in particular be used in the manufacture of a cosmetic, pharmaceutical or perfumery product.

An embodiment of the disclosure is in particular a composition according to embodiments of the disclosure as defined above, said composition being a colored, colorless and/or transparent product chosen from the following products:
makeup products for the human face and body, such as foundation, tinted cream, loose or compact powder, eye shadow, mascara, eyeliner, lipstick or nail varnish;
care products for the human face and body, such as cream, milk, lotion, mask, scrubbing product, cleansing and/or makeup-removing products, deodorants, antiperspirants, shaving products or hair-removing products;
hair products, such as shampoos, products for the shaping of the hair, products for retaining the hairstyle, antidandruff products, products for combating hair loss, products for combating dryness of the hair, hair dyes or bleaching products;
perfumery products, such as fragrance, milk, cream, or loose or compact scented powder.

### EXAMPLES

The examples below illustrate the disclosure without limiting the scope thereof. Unless otherwise indicated, all the percentages and parts are by weight.

PEBA 1 used: copolymer comprising PA12 blocks and PTMG blocks with respective molar masses in g/mol (600-2000).

The alcohol used is ethanol.

The following cosolvents are used:

Cosolvent 1: 2-ethylhexyl 2-cyano-3,3-diphenylacrylate

Cosolvent 2: ethylhexyl salicylate

Cosolvent 3: isopropyl lauroyl sarcosinate

Each of these cosolvents is miscible with the ethanol.

### Comparative 1 (Without Cosolvent):

PEBA 1 (5 parts by weight) is dissolved in ethanol (100 parts by weight) under hot conditions, 80° C. This solution gels under cold conditions, temperature within the range from 15 to 25° C.

### Comparative 2 (Without Ethanol):

The three cosolvents (1, 2 and 3) are tested.

5% by weight of 2533 are dispersed in each pure cosolvent, under ambient conditions (left to stir for up to 24 h at least at ambient temperature 15-25° C.) or by heating at 80° C. (close to the boiling point of the ethanol) until dissolved or at most one day.

Results for dissolution of the PEBA 1 granules in the various solvents:

Cosolvents 2 and 3:

10 g of solvent and 5% by weight of PEBA 1 granules, i.e. 0.5 g, are introduced into a 60 ml flask.

The flask is provided with a magnetic bar. The assembly is placed on a magnetic stirrer without heating.

After stirring for 8 hours, the granules are not dissolved.

Cosolvent 1:

This solvent is too viscous to be able to be stirred; the procedure is thus modified. In this case, use is made of a 250 ml round-bottom flask provided with a reflux condenser and a magnetic bar; the assembly is placed on a heating magnetic stirrer. 10 g of solvent and 5% of PEBA 1 granules are introduced. The temperature is gradually raised in order to have a stirrable medium. A temperature of 100° C. is sufficient to be able to stir the medium. After 5 h 00, the granules are dissolved. However, under cold conditions (15-25° C.), a gel is formed.

In the following tests (table 1: Cp3 and following tests), the operation is carried out with stirring (200 rev/min). Use is made of an oil bath to control the temperature and a conventional glass tube setup (small tube). The mixture is heated at reflux.

### Comparative 3 with the Pure Cosolvent 1 (Cp3):

20 g of solvent are placed in the glass tube to which 5% by weight of PEBA 1 granules are added: i.e. 1 g.

T=0 the tube is immersed.

T=20 min the granules begin to swell; the set temperature is 110° C.

T=240 min a gelatinous mixture is obtained.

Tests with Ethanol and Cosolvent (Cp 4 to 11 and Ex 1 to 4):

In these tests, a cosolvent/ethanol mixture is used as solvent. Different ratios by volume are tested. 5 parts by weight of PEBA 1 are added per 100 parts of total solvent. The set temperature is 80° C. (boiling point of the ethanol). Starting from a mixture comprising 60% of cosolvent, the temperature is increased by 10° C. in order to dissolve the PEBA 1 granules.

These tests and their results are collated in the following table 1. The columns under “Cosolvent 1”, “Cosolvent 2” and “Cosolvent 3” show the content of cosolvent (% by volume), with respect to the total volume of solvent, the remainder of solvent being the ethanol.

Results:

In order to obtain a liquid solution which is stable under cold conditions, that is to say at ambient temperature (15-25° C.), it is necessary to use at least 15% (Cp6), indeed even 20% (Cp10), of cosolvent with the ethanol. The cosolvents 1, 2 and 3, starting from 51% by volume in the total solvent, give a clear liquid solution at ambient temperature. From table 1, it is found in particular that:

For 60% of cosolvent 1, a clear liquid solution is obtained which is stable at ambient temperature.

For 51% of cosolvent 2, a clear liquid solution is obtained which is stable at ambient temperature.

For 60% of cosolvent 3, a clear liquid solution is obtained which is stable at ambient temperature.

For 15-50% of cosolvent, the solution remains liquid at ambient temperature but is opaque.

TABLE 1

| | | | | State of the | Appearance of |
|---|---|---|---|---|---|
| | | | | product | the product |
| | Cosolvent | Cosolvent | Cosolvent | under cold | under cold |
| Test | 1 | 2 | 3 | conditions | conditions |
| Cp3 | 100% | | | gel | opaque |
| Ex1 | 60% | | | liquid | clear |
| Cp4 | 50% | | | very viscous | clear |
| Cp5 | 40% | | | liquid | opaque |
| Ex2 | | 60% | | liquid | clear |
| Ex3 | | 51% | | liquid | clear |
| Cp6 | | 20% | | liquid | opaque |
| Cp7 | | 10% | | gel | opaque |
| Ex4 | | | 60% | viscous | clear |
| | | | | liquid | |
| Cp8 | | | 50% | viscous | opaque |
| | | | | liquid | |
| Cp9 | | | 30% | liquid | opaque |
| Cp10 | | | 15% | liquid | opaque |
| Cp11 | | | 5% | gel | very opaque |

With the tests of the examples in accordance with the disclosure, Ex1, Ex2, Ex3 and Ex4, the product obtained is used to form a film. A layer of product is spread over any support: metal, wood, plastic, nail, skin. The solvent is evaporated. A film is obtained which, on contact with water, withstands the water and remains attached to the support.

1. A liquid composition comprising from 1 to 15 parts by weight of PEBA per 100 parts by weight of solvent, wherein: said solvent comprises from 1% to 49% by volume of light C₁ to C₆ alcohol and from 51% to 99% by volume of a cosolvent which is miscible with the alcohol, with respect to the total volume of solvent.

2. The composition as claimed in claim 1, wherein the cosolvent is chosen from fatty acids, fatty esters, fatty alcohols, fatty ethers and their mixtures.

3. The composition as claimed in claim 1, wherein the composition is in the form of a solution which is clear at a temperature of 15 to 25° C.

4. The composition as claimed in claim 1, wherein the composition comprises from 1 to 10 parts, of PEBA, per 100 parts of solvent.

5. The composition as claimed in claim 1, in which the PEBA comprises more than 50% by weight of polyether blocks with respect to the total weight of PEBA.

6. The composition as claimed in claim 1, wherein the PEBA comprises polyether blocks composed predominantly based on PTMG.

7. The composition as claimed in claim 1, wherein the composition additionally comprises from 1 to 10 parts of additive chosen from: UV screening agents, antioxidants, pigments, fillers, or cosmetic or pharmaceutical active agents.

8. A product comprising the composition as claimed in claim 1, wherein the product has at least one of the following forms: dispersion, solution, emulsion, microemulsion, nanoemulsion, dry emulsion, suspension, aerosol, gel, gum, plastic gum, paste, foam, cream, powder, butter, film, elastic film and their mixtures or combinations.

9. A manufacture comprising the product as claimed in claim 8, wherein the manufacture is a water-resistant objects selected from coatings, films, varnishes, paints, individual protective equipment, gloves, flexible masks, condoms or protective accessories for electronic or computing equipment.

10. The product as claimed in claim 8, wherein said product is manufactured by at least one of the following methods: spreading, spraying, dipping, evaporation of the solvent from the composition.

11. A manufacture comprising the product as claimed in claim 8, wherein the manufacture is selected from a cosmetic, pharmaceutical or perfumery product.

12. The composition as claimed in claim 1, said composition being a colored, colorless and/or transparent product chosen from the following products:
makeup products for the human face and body;
care products for the human face and body;
hair products;
perfumery products.
